(19) United States
(12) Patent Application Publication
(10) Pub. No.: US 2026/0174446 A1
(43) Pub. Date: Jun. 25, 2026

(54) DEVICE FOR OPERATING ON A PEDICLE WITHIN A PATIENT

(71) Applicant: PANONI'S B.V., Amsterdam (NL)

(72) Inventor: Micha Ilan Paalman, Amsterdam (NL)

(21) Appl. No.: 19/126,803

(22) PCT Filed: Oct. 16, 2023
(86) PCT No.: PCT/NL2023/050539

§ 371 Date: May 2, 2025

(30) Foreign Application Priority Data
Nov. 3, 2022 (NL) 2033451

(51) Int. Cl. A61B 17/17; A61B 6/00; A61B 17/16; A61B 17/34; A61B 17/70; A61B 17/86; A61B 17/88

(52) U.S. Cl. CPC A61B 17/1757; A61B 6/4405; A61B 17/1671; A61B 17/7032; A61B 17/705; A61B 17/7082; A61B 17/86; A61B 17/8866; A61B 2017/3411

(57) ABSTRACT

A device for operating on a pedicle within a patient, said device comprising a guide portion comprising a guide channel extending longitudinally therealong for guiding a tool to the pedicle, such as a needle or a bore, and said device, further comprising a handle which is mounted or mountable to the guide portion wherein the device is further equipped with a sleeve which snugly and removably fits around the guide portion, and which sleeve has a resiliently openable nose or forward end through which the guide portion is extendable by retracting the sleeve whilst the guide portion is received in the sleeve.

### CROSS-REFERENCE TO RELATED APPLICATION(S)

The present application is a § 371 national phase entry of International patent application Serial No. PCT/NL2023/050539, filed Oct. 16, 2023, and published in English, and claims priority from Netherlands application no. 2033451 filed on Nov. 3, 2022.

### BACKGROUND

The invention relates to a device for operating on a pedicle within a patient, said device comprising a guide portion comprising a guide channel extending longitudinally therealong for guiding a tool to the pedicle, such as a needle or a bore, and said device further comprising a handle which is mounted or mountable to the guide portion.

Pedicles of the vertebrae are the short, thick, cylindrical bony parts that project posteriorly from the superior part of the vertebral body.

US2003/0236447 discloses a device adapted for placement of a pedicle screw within a patient, comprising:
a guide portion having a guide channel extending longitudinally therealong, said guide portion including a first end, a second end, and a tool groove;
a handle connected angularly with respect to said first end of said guide portion; wherein the handle provides appropriate holding leverage to maintain the guide so as to stabilize the guide against the lateral aspects of the facet of the pedicle with the paraspinous musculature; and
an insert configured to fit within said guide channel, said insert having an aperture extending lengthwise therethrough, said aperture having a diameter sized to accommodate a tool being inserted therethrough.

The guide channel has an appropriate size to accommodate a variety of tools, including a drill bit or a tap. The channel also provides a pathway for a probe or feeler to inspect the placement of the screw. A tool groove may be provided in the channel to support the shaft of a tool, such as a tap.

There are many inaccuracies and problems reported in prior art minimal invasive operations on pedicles. Reference is made to the article by Zeng et al. Eur J Med Res (2015) 20:80 entitled Analysis of risk factors for adjacent superior vertebral pedicle-induced facet joint violation during the minimally invasive surgery transforaminal lumbar interbody fusion: a retrospective study.

Further reference is made to the article by Moon-Chan Kim et al. Eur Spine J (2011) 20:1635-1643 entitled Factors affecting the accurate placement of percutaneous pedicle screws during minimally invasive transforaminal lumbar interbody fusion. This article reports that age, gender, body mass index, bone mineral density, diagnosis, operation time, estimated blood loss (EBL), level of fusion, surgeon's position, spinal alignment, quality/quantity of multifidus muscle, and depth to screw entry point were considered to be demographic and anatomical variables capable of affecting pedicle screw placement. Pedicle dimensions, facet joint arthritis, screw location (ipsilateral or contralateral), screw length, screw diameter, and screw trajectory angle were regarded as screw-related variables that affect pedicle screw placement.

### SUMMARY

It is an object of the invention to simplify the design of the known device, to make it more accurate in placing the pedicle screw or other tool, and to make it easier and more transparent for the operator/surgeon to manipulate during operation on the pedicle.

In a particular aspect the invention departs from the prior art in that it no longer requires the use of an insert fitting in the guide channel of the guide portion. In the invention the device is rather provided with the features of one or more of the appended claims.

The device is equipped with a sleeve which removably fits around the guide portion, and which sleeve has a resiliently openable nose or forward end through which the guide portion is extendable by retracting the sleeve whilst the guide portion is received in the sleeve. The resiliently openable nose arranges that the nose will close upon retraction of the guide portion.

The forward end of the sleeve is convex as a fingertip when the guide portion does not extend through said forward end. This enables a smooth insertion into the skin incision and enables the operator, the surgeon, to feel the edges of the pedicle bone to determine the right position of the sleeve on the pedicle bone before it is retracted for placing the guide portion on this proper position on the pedicle bone.

This enables that the sleeve can first accurately be placed in position against the pedicle, which implies that also the guide portion within the sleeve is in the correct position. Thereafter the sleeve can be retracted whilst the guide portion remains stationary, thus without adversely affecting the position of the guide portion that then comes to extend through the resiliently openable forward end of the sleeve so as to engage the pedicle.

The device further has several beneficial features. For re-use of the handle and other advantages it is preferable that the handle and the guide portion are removable from each other. Of course it is also possible that the handle and the guide portion form an integral unit. Further, the ease of handling during operation on the pedicle is greatly promoted by arranging that a blunt angle exists between the handle and the guide portion to accommodate an operator to stably maintain the guide portion in a desired orientation and position with reference to the pedicle.

Preferably the sleeve is provided with sidewards extending finger handles to enable an operator to retract the sleeve with his fingers.

To ensure a reliable placement of the guide portion with reference to the pedicle bone, it is the desirable that the sleeve and the guide portion snugly fit to each other and have a matching internal and external shape, respectively. Preferably the matching internal and external shape is a rectangular shape.

It is further preferred that at or near its forward end the guide portion is provided with a navigational tool. This enables an accurate positioning of the tool inside the guide portion. Preferably the navigational tool is an element that is x-ray detectable.

Suitably the navigational tool is embodied as a ring or a number of rings which is/are x-ray detectable.

It is further preferred that the guide portion has an extremity at its forward end that is shaped to accommodate a stable positioning of the guide portion on the pedicle. Preferably the extremity is V-shaped. The extremity provides a so-called dock for accurate placement of the guide portion on the pedicle bone.

Advantageously the guide portion comprises two or more guide channels that are at least partly separated from each other. This allows the operator, the surgeon, to choose one of the guide channels for a particular slightly off-center placement of the tool or instrument that is guided through the guide portion to and eventually into the pedicle.

### BRIEF DESCRIPTION OF THE DRAWINGS

The accompanying drawing, which is incorporated into and forms a part of the specification, illustrates one or more embodiments of the present invention and, together with the description, serves to explain the principles of the invention. The drawing is only for the purpose of illustrating one or more embodiments of the invention and is not to be construed as limiting the invention.

In the drawing:

FIGS. 1A/1B provide different views at a device being brought in proximity of a to be treated pedicle;

FIG. 2 provides different views at a sleeve forming part of a device;

FIG. 3 provides different views at a guide portion forming part of a device;

FIGS. 4A-4D provides different views at the device being placed in position on the pedicle to be treated;

FIGS. 5A-5D provides different views at the device wherein the sleeve is retracted from the guide portion, whilst being held in position on the pedicle to be treated;

FIG. 6A/6B provide different views at the device while a needle or bore is guided through the guiding portion to the pedicle to be treated;

FIG. 7A/7B provide different views at the device while placing a K wire in the pedicle to be treated; and

FIGS. 9A-9C provides several views at a frontal portion of the guide portion of the device, depicting the application of one or more guide channels.

Whenever in the figures the same reference numerals are applied, these numerals refer to the same parts.

### DETAILED DESCRIPTION OF ILLUSTRATIVE EMBODIMENTS

FIG. 1A/1B shows the device 1 for operating on a pedicle 2 within a patient. FIG. 1A shows the skin 10 of the patient. The figures do not show the other pedicles of the spine of the patient, but this is common knowledge to the skilled person and requires no elucidation. Moreover the patient does not form part of the invention and plays therefore no role in disclosing the invention in a manner sufficiently clear and complete to enable the skilled person to work and design a device according to the invention.

The device 1 comprises a guide portion 3 comprising an internal guide channel extending longitudinally along the guide portion 3 for guiding a tool to the pedicle 2, such as a needle or a bore. This is also known to the skilled person and therefore not shown.

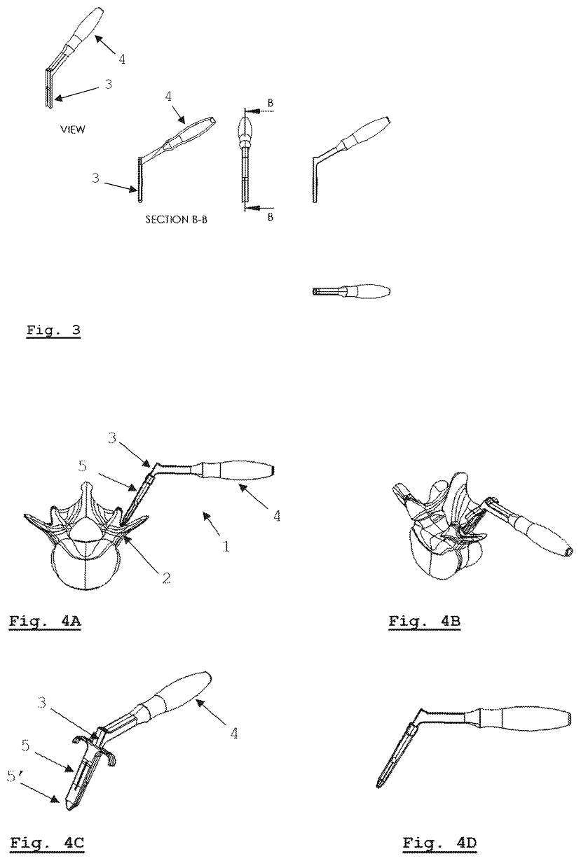

The device 1 further comprises a handle 4 which is either integral with or mounted or mountable to the guide portion 3. The guide portion 3 and the handle 4 are together separately shown from the other elements of the device 1 in FIG. 3.

FIG. 3 provides a view at the guide portion 3 and the handle 4 from different angles.

Preferably the handle 4 and the guide portion 3 are removable from each other. It shows further in FIG. 1A/1B and FIG. 3 that a blunt angle exists between the handle 4 and the guide portion 3 which is intended to accommodate an operator during use of the device 1 to stably orient and position the guide portion 3 with reference to the pedicle 2 to be treated.

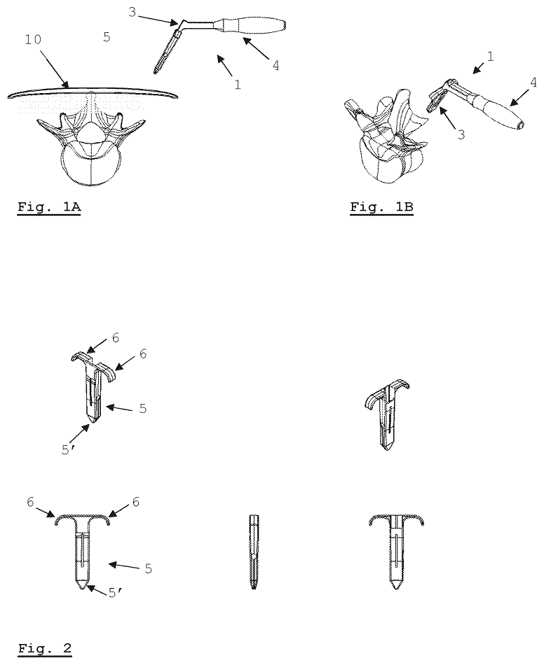

The device 1 is further equipped with a sleeve 5 that is separately shown from the other features of the device 1 in FIG. 2. FIG. 2 provides a view at the sleeve 5 from different angles.

It has to be noted that preferably the sleeve 5 snugly and removably fits around the guide portion 3.

In a first operational step following the point where the device 1 approaches the pedicle 2 to be treated as shown in FIG. 1A/1B, the device 1, that is to say the nose or forward end 5′ of the sleeve portion 5 is placed on the pedicle 2. This is depicted in FIGS. 4A-4D. FIG. 4A/4B show the device 1 at the pedicle 2. FIG. 4C/4D show the device 1 without the pedicle 2. In connection with the first operational step it is noted that the nose or forward end 5′ of the sleeve 5 is convex as a fingertip.

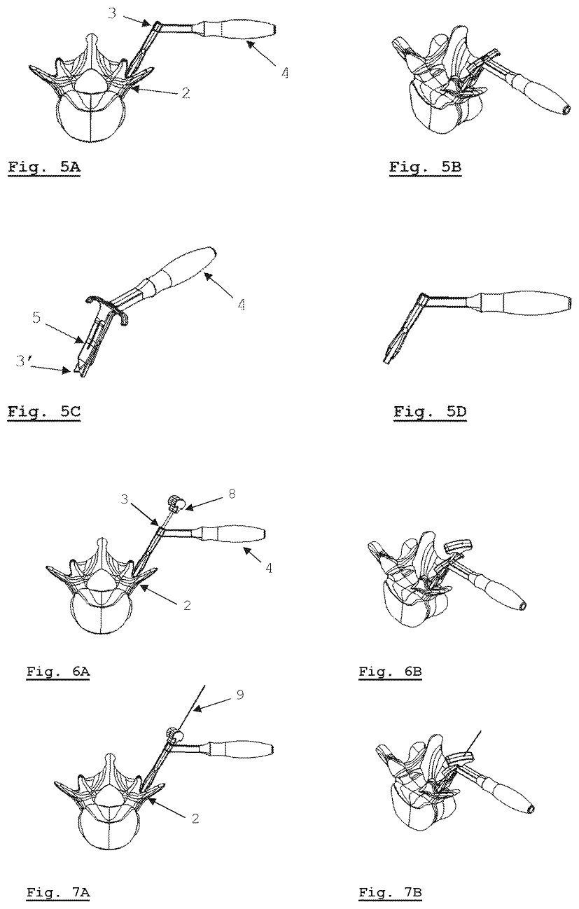

A next step is depicted in FIGS. 5A-5D. FIG. 5A/5B show the device 1 at the pedicle 2. FIG. 5C/5D show the device 1 without the pedicle 2. In connection with this next step it is noted that the sleeve 5 has a resiliently openable nose or forward end 5′ through which the guide portion 3 is extendable by retracting the sleeve 5 whilst the guide portion 3 is received in the sleeve 5 and pushed to the pedicle 2. For retracting the sleeve 5, the sleeve 5 is provided with sidewards extending finger handles 6 to enable an operator/surgeon to retract the sleeve 5 with his fingers, whilst at the same time maintaining the position of the guide portion 3 with his thumb, It is further noted that the sleeve 5 and the guide portion 3 have a matching internal and external shape, respectively so that the sleeve 5 and the guide portion 3 are rotationally fixed around their longitudinal axis. Preferably the matching internal and external shape is a rectangular shape. It is also possible to secure that the sleeve 5 and the guide portion 3 are rotationally fixed by providing these parts with a key and a keyway that cooperates with the key, in particular when the sleeve 5 and the guide portion 3 have a circular cross section.

Particularly the FIGS. 5C and 5D provide a clear view at the guide portion 3 extending through the nose or forward end 5′ of the sleeve 5 following the retraction of the sleeve 5. By this operation the guide portion 3 comes to directly engage the pedicle bone 2 on the intended location which is previously established by the operator/surgeon when placing the sleeve 5 on the pedicle bone. FIG. 5C clearly depicts that the guide portion 3 has a V-shaped extremity at its forward end. This provides a stable positioning on the pedicle 2 to be treated.

It is noted that during placement of the sleeve 5, or the engagement of the pedicle bone by the guide portion 3, the proper location of the tool of the device 1 on the pedicle 2 can be established using a navigational tool which is provided at or near the forward end the guide portion 3. The navigational tool can have any form or shape, and is visible in FIG. 9A to be discussed hereinafter, but it is anyway clear for the skilled person how this could be applied. In a preferable embodiment the navigational tool is an element that is x-ray detectable such as a ring 7 as shown in FIG. 9A which is x-ray detectable, preferably a metal ring. As said the form or shape of the navigational tool can be selected depending on the situation; instead of being circular as a ring it may also have any other suitable shape. Also the number of rings can be varied in order to improve the accuracy of establishing the location of the tool being advanced in the guide portion 3.

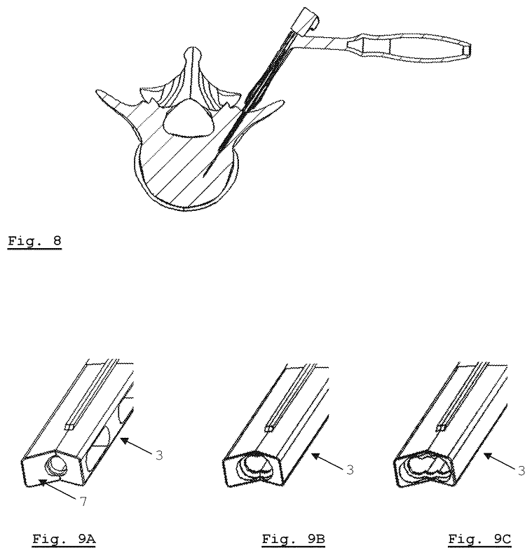

After the correct positioning and placement of the device 1 on the pedicle 2 is completed as depicted in FIGS. 5A-5D, a needle or bore 8 can be entered into the guiding channel of the guide portion 3 as shown in FIG. 6A/6B, after which the needle or bore 8 can be removed and a K wire 9 can be placed in the pedicle 2 as depicted in FIG. 7A/7B and FIG. 8.

Finally reference is made to FIGS. 9A-9C which show a frontal part of the guide portion 3, and illustrates that the guide portion 3 may be provided with a single guide channel (FIG. 9A), a dual guide channel (FIG. 9B), or a triple guide channel (FIG. 9C) It is noted that the two or more guide channels are at least partly separated from each other.

Embodiments of the present invention can include every combination of features that are disclosed herein independently from each other. Although the invention has been discussed in the foregoing with reference to an exemplary embodiment of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another.

1. A device for operating on a pedicle within a patient, said device comprising a guide portion comprising a guide channel extending longitudinally therealong for guiding a tool to the pedicle and said device further comprising a handle which is mounted or mountable to the guide portion, which device is further equipped with a sleeve which removably fits around the guide portion, and which sleeve has an openable nose or forward end through which the guide portion is extendable by retracting the sleeve whilst the guide portion is received in the sleeve, wherein the nose or forward end of the sleeve is resiliently openable to arrange that the nose will close upon retraction of the guide portion, and said forward end of the sleeve is convex as a fingertip when the guide portion does not extend through said forward end, wherein the guide portion has an extremity at its forward end that is shaped to accommodate a stable positioning of the guide portion on the pedicle.

2. The device of claim 1, wherein the handle and the guide portion are removable from each other.

3. The device of claim 2, wherein a blunt angle exists between the handle and the guide portion to accommodate an operator to stably orient and position the guide portion with reference to the pedicle.

4. The device of claim 3, wherein the sleeve is provided with sidewards extending finger handles to enable an operator to retract the sleeve with his fingers.

5. The device of claim 4, wherein the sleeve and the guide portion have a snugly fit with respect to each other with a matching internal and external shape, respectively.

6. The device of claim 5, wherein the sleeve and the guide portion have a matching key and keyway.

7. The device of claim 5, wherein the matching internal and external shape is a rectangular shape.

8. The device of claim 7, wherein at or near its forward end the guide portion is provided with a navigational tool.

9. The device of claim 8, wherein the navigational tool is an element that is x-ray detectable.

10. The device of claim 8, wherein the navigational tool comprises a ring or multiple rings which is or are x-ray detectable.

11. The device of claim 1, wherein the extremity is V-shaped.

12. The device of claim 1, wherein the guide portion comprises two or more guide channels that are at least partly separated from each other.

13. The device of claim 1, wherein a blunt angle exists between the handle and the guide portion to accommodate an operator to stably orient and position the guide portion with reference to the pedicle.

14. The device of claim 1, wherein the sleeve is provided with sidewards extending finger handles to enable an operator to retract the sleeve with his fingers.

15. The device of claim 1, wherein the sleeve and the guide portion have a snugly fit with respect to each other with a matching internal and external shape, respectively.

16. The device of claim 15, wherein the sleeve and the guide portion have a matching key and keyway.

17. The device of claim 15, wherein the matching internal and external shape is a rectangular shape.

18. The device of claim 1, wherein at or near its forward end the guide portion is provided with a navigational tool.

19. The device of claim 18, wherein the navigational tool is an element that is x-ray detectable.

20. The device of claim 18, wherein the navigational tool comprises a ring or multiple rings which is or are x-ray detectable.
